(19) United States
(12) Patent Application Publication
(10) Pub. No.: US 2025/0235224 A1
(43) Pub. Date: Jul. 24, 2025

(54) THROMBOLYSIS PROMOTING MODULE AND INTERVENTIONAL THROMBUS REMOVAL DEVICE

(71) Applicant: Beijing HeQingHeChuang Medical Technology Co., Ltd., Beijing (CN)

(72) Inventors: Haosheng CHEN, Beijing (CN); Yunfan PAN, Beijing (CN); Yongjian LI, Beijing (CN)

(21) Appl. No.: 18/703,612

(22) PCT Filed: Oct. 17, 2022
(86) PCT No.: PCT/CN2022/125679

§ 371 Date: Apr. 22, 2024

(30) Foreign Application Priority Data
Oct. 22, 2021 (CN) 202111232878.4

(51) Int. Cl. A61B 17/22; A61B 17/00

(52) U.S. Cl. CPC A61B 17/2202; A61B 17/00234; A61B 2017/00146; A61B 2017/00238; A61B 2017/00305; A61B 2017/22008

(57) ABSTRACT

A thrombolysis promoting module and an interventional thrombus removal device. The thrombolysis promoting module includes a driving module and a cavitation module. The driving module is an ultrasonic module for generating acoustic energy. The driving module is configured to generate ultrasonic waves with a first frequency in a circumferential direction. The cavitation module is an ultrasonic module for generating acoustic energy. The cavitation module is configured to generate ultrasonic waves with a second frequency greater than the first frequency in the circumferential direction. The thrombolysis promoting module of the present disclosure can drive the microbubble precursors and the thrombolytic drug to penetrate into the thrombus, and can induce cavitation in the microbubble precursors that penetrate into the thrombus or on surfaces of the microbubble precursors to form microbubbles.

### RELATED APPLICATION

The present disclosure claims priority to Chinese Invention patent application No. 202111232878.4 filed on Oct. 22, 2021, which is hereby incorporated by reference in its entirety.

### TECHNICAL FIELD

The present disclosure relates to the technical field of medical instruments, and particularly to a thrombolysis promoting module and an interventional thrombus removal device.

### BACKGROUND

The cardiovascular and cerebrovascular embolism is one of the main diseases that endanger human life and health, especially the deep venous thrombosis (DVT), which greatly affects the health and living quality of patients. At present, the conventional clinical methods for the treatment of thrombus include drug thrombolysis, vascular stent, mechanical rotary cutting, ultrasonic thrombolysis, etc. However, these methods all have some defects. For example, the drug thrombolysis is easy to cause complications such as bleeding, and the mechanical rotary cutting is easy to cause vascular injury. Therefore, there is an urgent need for a safer and more efficient interventional thrombus removal device.

### SUMMARY

In order to solve the above problems pointed out in the background, embodiments of the present disclosure provide a thrombolysis promoting module and an interventional thrombus removal device.

Embodiments of a first aspect of the present disclosure provide a thrombolysis promoting module, which includes a driving module and a cavitation module. The driving module is an ultrasonic module for generating acoustic energy. The driving module is configured to generate ultrasonic waves with a first frequency in a circumferential direction. The ultrasonic waves with the first frequency is capable of driving microbubble precursors to penetrate into a thrombus. The cavitation module is an ultrasonic module for generating acoustic energy. The cavitation module is configured to generate ultrasonic waves with a second frequency in the circumferential direction. The second frequency is greater than the first frequency. The ultrasonic waves with the second frequency is capable of inducing cavitation in the microbubble precursors that penetrate into the thrombus, or inducing cavitation on surfaces of the microbubble precursors, to form microbubbles.

Embodiments of a second aspect of the present disclosure provide another thrombolysis promoting module, which includes a driving module and a cavitation module. The driving module is an ultrasonic module for generating acoustic energy. The driving module includes one or a plurality of first piezoelectric elements. The cavitation module is an ultrasonic module for generating acoustic energy. The cavitation module includes one or a plurality of second piezoelectric elements insulated from the first piezoelectric element. The driving module is configured to generate ultrasonic waves with a first frequency in a circumferential direction. The cavitation module is configured to generate ultrasonic waves with a second frequency in the circumferential direction, and the second frequency is greater than the first frequency.

Embodiments of a third aspect of the present disclosure provide an interventional thrombus removal device, which includes at least one thrombolysis promoting module according to the aforementioned embodiments and a main catheter. The main catheter defines a lumen and includes a distal part for accommodating the thrombolysis promoting module. The distal part is configured to release microbubble precursors in the lumen to the outside of the main catheter.

The advantageous effects of the embodiments of the present disclosure include: by disposing the driving module which generates the first energy, it is possible to drive the microbubble precursors and the thrombolytic drug to penetrate into the thrombus, so as to expand the action range of the thrombolytic drug on the thrombus and promote thrombolysis; by disposing the cavitation module which generates the second energy, it is possible to induce cavitation in the microbubble precursors that penetrate into the thrombus or induce cavitation on the surfaces of the microbubble precursors, to form microbubbles which loosen the thrombus, thereby increasing the contact area between thrombolytic drug and the thrombus, further promoting thrombolysis, and effectively improving the thrombolytic efficiency, which is safe and efficient.

### BRIEF DESCRIPTION OF DRAWINGS

The drawings are included to provide a further understanding of the embodiments of the present disclosure, and constitute a part of the specification, to illustrate the embodiments of the present disclosure, and to explain the principles of the present disclosure together with the description. Obviously, the drawings involved the following description only illustrate some embodiments of the present disclosure. For those of ordinary skill in the art, other drawings can be derived from these drawings without any inventive efforts. In the drawings:

FIG. 1 illustrates a schematic diagram of a thrombolysis promoting module according to an embodiment of the present disclosure;

FIG. 2 illustrates a cross-sectional view of an example taken along line A-A in FIG. 1;

FIG. 3 illustrates a cross-sectional view of an example taken along line B-B in FIG. 1;

FIG. 4 illustrates a cross-sectional view of another example taken along line A-A in FIG. 1;

FIG. 5 illustrates a cross-sectional view of another example taken along line B-B in FIG. 1;

FIG. 6 illustrates a schematic diagram of a thrombolysis promoting module according to another embodiment of the present disclosure;

FIG. 7 illustrates a cross-sectional view of an example taken along the line C-C in FIG. 6;

FIG. 8 illustrates a cross-sectional view of an example taken along the line D-D in FIG. 6;

FIG. 9 illustrates a schematic diagram of a working principle of a thrombolysis promoting module in a driving stage according to an embodiment of the present disclosure;

FIG. 10 illustrates a schematic diagram of a working principle of a thrombolysis promoting module in a cavitation stage according to an embodiment of the present disclosure;

FIG. 11 illustrates a working timing diagram of a thrombolysis promoting module according to an embodiment of the present disclosure;

FIG. 12 illustrates a schematic diagram of an interventional thrombus removal device according to an embodiment of the present disclosure; and

FIG. 13 illustrates a schematic diagram of an interventional thrombus removal device according to another embodiment of the present disclosure.

### DETAILED DESCRIPTION OF EMBODIMENTS

For a clearer understanding of the objectives, technical features and effects of the embodiments of the present disclosure, specific embodiments will now be described with reference to the drawings. The described embodiments are intended only to schematically illustrate and explain this invention and do not limit the scope of the present disclosure.

In the embodiments of the present disclosure, the terms “first”, “second”, etc. are used to distinguish different elements in terms of name, but they do not mean a spatial arrangement, a temporal order, or the like of these elements, and these elements should not be limited by the above terms. The term “and/or” includes any and all combinations of one or more of the associated listed terms. The terms “include”, “comprise”, “have”, etc. refer to the presence of the stated features, elements, members or components, but do not exclude the presence or addition of one or more other features, elements, members or components.

In the embodiments of the present disclosure, a singular form “a”, “the”, or the like may include a plural form, and should be broadly understood as “one type” or “one class” and not limited to the meaning of “one”. In addition, the term “said” should be understood to include both singular and plural forms, unless otherwise specified in the context. Moreover, the term “according to” should be understood as “at least in part according to . . . ” and the term “based on” should be understood as “at least in part based on . . . ”, unless otherwise specified in the context. Further, the term “a plurality of” means two or more unless otherwise specified.

The implementations of the embodiments of the present disclosure will be described below with reference to the drawings.

Embodiments of a first aspect of the present disclosure provide a thrombolysis promoting module 10.

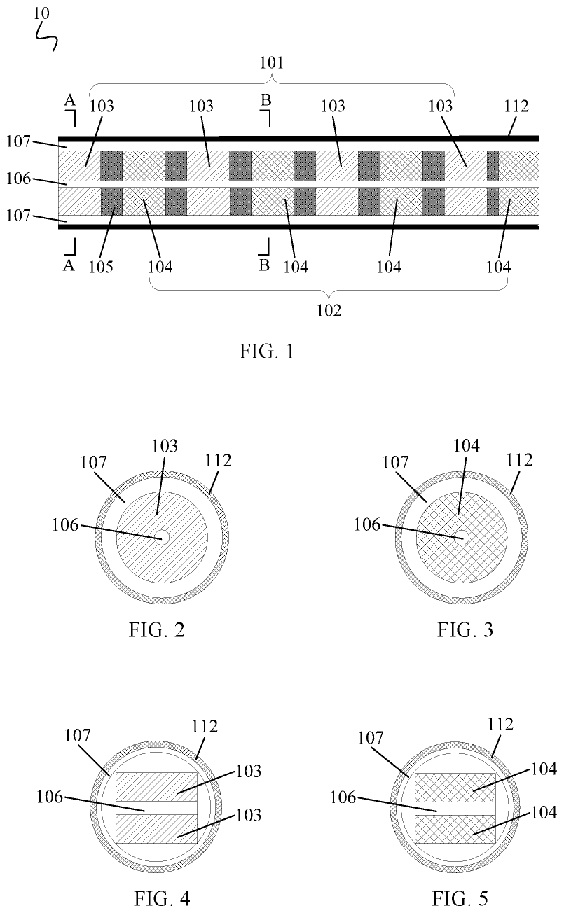

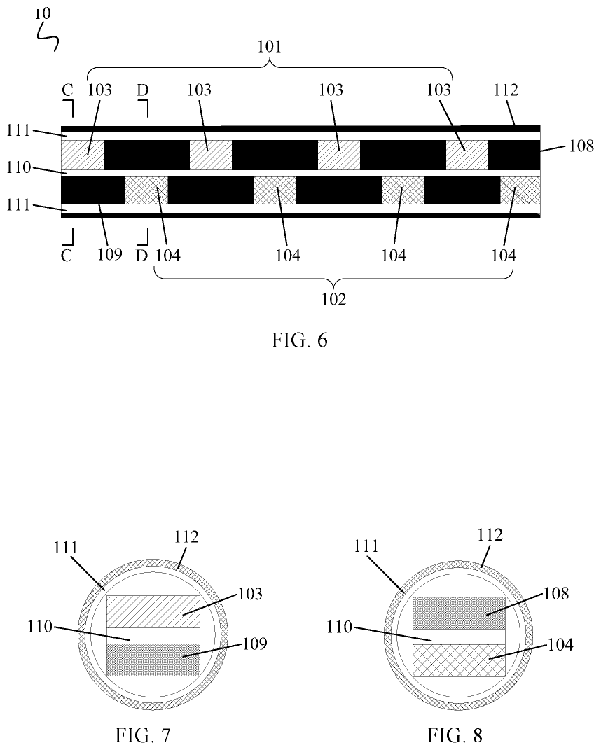

FIG. 1 illustrates a schematic diagram of a thrombolysis promoting module 10 according to an embodiment of the present disclosure, and FIG. 6 illustrates a schematic diagram of a thrombolysis promoting module according to another embodiment of the present disclosure.

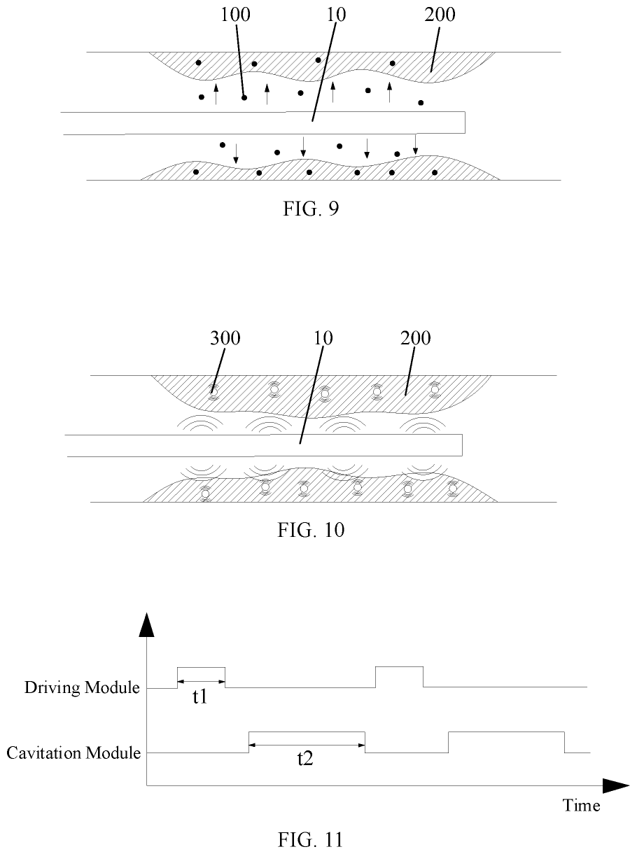

As illustrated in FIGS. 1 and 6, the thrombolysis promoting module 10 according to the embodiments of the present disclosure is used for interventional thrombus removal, and includes a driving module 101 and a cavitation module 102. The driving module 101 is configured to generate first energy for driving microbubble precursors 100 to penetrate into a thrombus 200 (as illustrated in FIG. 9). The microbubble precursors 100 are injected into a patient's blood vessel together with an thrombolytic drug during an interventional thrombolytic treatment, and the microbubble precursors 100 and the thrombolytic drug are in a mixed state. Thus, the microbubble precursors 100 and the thrombolytic drug (not illustrated) are driven by the first energy to penetrate into the thrombus 200, thereby increasing an action range of the thrombolytic drug on the thrombus 200 and promoting thrombolysis. The cavitation module 102 is configured to generate second energy (as illustrated in FIG. 10) for inducing cavitation in the microbubble precursors 100 that penetrate into the thrombus 200 or inducing cavitation on surfaces of the microbubble precursors, and the second energy is different from the first energy. The microbubble precursors 100 absorb the second energy and is locally cavitated to form microbubbles 300 which loosen the thrombus 200, thereby increasing the contact area between the thrombolytic drug and the thrombus 200, and further promoting thrombolysis and improving the efficiency of thrombus removal.

During use of the thrombolysis promoting module 10 according to the embodiment of the present disclosure, as illustrated in FIG. 11, the driving module 101 may be started first to drive the microbubble precursors 100 and the thrombolytic drug to penetrate into the thrombus 200, which may be called as a driving stage; after the driving stage lasts for a preset time t1, the cavitation module 102 is started to cavitate the microbubble precursors 100 that penetrate into the thrombus 200 to form the microbubbles 300, which may be called as a cavitation stage; after the cavitation stage lasts for a preset time t2, one thrombolysis process is completed. In order to enhance the thrombolysis effect, the above thrombolysis process may be carried out several times, i.e., the driving stage and the cavitation stage may be alternately carried out many times. In some embodiments, at least one selected from the driving module 101 and the cavitation module 102 may be configured as an ultrasonic module for generating acoustic energy, i.e., the first energy and/or the second energy is acoustic energy, which is safe and efficient.

The present disclosure is not, however, limited thereto. In some other embodiments, at least one selected from the driving module 101 and the cavitation module 102 may be configured as a heating element for generating heat energy or a light emitting element for generating light energy, i.e., the first energy and/or the second energy may be heat energy or light energy.

In some embodiments, the driving module 101 and the cavitation module 102 may both be configured as ultrasonic modules for generating acoustic energy, the first energy is ultrasonic waves with a first frequency, and the second energy is ultrasonic waves with a second frequency that is different from the first frequency.

Exemplarily, the second frequency is greater than the first frequency, i.e., ultrasonic waves with a lower frequency serves as the first energy to drive the microbubble precursors 100 to penetrate into the thrombus 200, and ultrasonic waves with a higher frequency serves as the second energy to cavitate the microbubble precursors 100 that penetrate into the thrombus 200 to form the microbubbles 300.

In some embodiments, the microbubble precursors 100 may be micro/nano-droplets, which can be cavitated into microbubbles. Correspondingly, the first frequency may be 20 kHz to 1 MHZ, and the second frequency may be 1 MHz to 20 MHz. For example, the micro/nano-droplets may be fluorocarbon droplets with a diameter of 100 nm to 800 nm.

In some other embodiments, the microbubble precursors 100 may be micro-nanoparticles, and gas cores at the interface between the micro-nanoparticles and the solution may be cavitated into microbubbles. Correspondingly, the first frequency may be 20 kHz to 1 MHz, and the second frequency may be 1 MHz to 20 MHz. For example, the micro/nano-particles may be porous nanospheres with a diameter of 10 nm to 500 nm.

In addition to the above embodiments, the microbubble precursors 100 may also be a mixture of micro/nano-droplets and micro/nano-particles. Those skilled in the art may select the morphology, composition and size of the microbubble precursors 100 as needed, and may determine the corresponding first and second frequencies. These changes, modifications and equivalents all fall within the protection scope of the present disclosure.

In some embodiments, as illustrated in FIGS. 1 and 6, the driving module 101 includes one or a plurality of first piezoelectric elements 103, and the cavitation module 102 includes one or a plurality of second piezoelectric elements 104. The first piezoelectric element 103 and the second piezoelectric element 104 are insulated from each other, so as to generate ultrasonic waves independently of each other without interfering with each other.

In order to provide excitation signals to the first piezoelectric element 103 and the second piezoelectric element 104, a positive electrode and a negative electrode may be provided for contacting the first piezoelectric element 103, and a positive electrode and a negative electrode may be provided for contacting the second piezoelectric element 104.

The first piezoelectric element 103 and the second piezoelectric element 104 may be made of a piezoelectric material, which may be, for example, lead zirconate titanate. The electrodes may be made of an electrically conductive material, which may be, for example, silver or copper.

In some embodiments, as illustrated in FIGS. 1 and 6, the driving module 101 includes a plurality of first piezoelectric elements 103, which are arranged at intervals in an axial direction and insulated from each other; the cavitation module 102 includes a plurality of second piezoelectric elements 104, which are arranged at intervals in the axial direction and insulated from each other.

In these embodiments, by arranging the plurality of first piezoelectric elements 103 and the plurality of second piezoelectric elements 104 at intervals in the axial direction, it is possible to generate ultrasonic waves at different positions in the axial direction, expand the energy action range, and further improve the thrombolytic efficiency. In some other embodiments, it is also possible to actuate only some of the piezoelectric elements according to actual needs, so as to generate ultrasonic waves at specific positions, thereby improving the flexibility and convenience of use.

In the example of FIG. 1, the plurality of first piezoelectric elements 103 and the plurality of second piezoelectric elements 104 are alternately arranged in the axial direction, and the adjacent first piezoelectric element 103 and second piezoelectric element 104 may be separated by an insulating element to achieve insulation.

The first piezoelectric element 103 and the second piezoelectric element 104 may be constructed as a variety of different structures.

For example, in a feasible technical solution, as illustrated in FIGS. 2 and 3, the first piezoelectric elements 103 and the second piezoelectric elements 104 are coaxially and alternately arranged, and each first piezoelectric element 103 and each second piezoelectric element 104 may be cyclic structures, such as annular structures, so as to generate ultrasonic waves in the whole circumferential range in the circumferential direction. The first piezoelectric element 103 and the second piezoelectric element 104 are separated by an insulating element 105 (as illustrated in FIG. 1). In this technical solution, an elongated first electrode 106 extending continuously in the axial direction may be disposed in central holes of the first piezoelectric element 103 and the second piezoelectric element 104, a second electrode 107 extending continuously in the axial direction may be disposed on outer peripheries of the first piezoelectric element 103 and the second piezoelectric element 104, and the polarity of the second electrode is opposite to that of the first electrode, so that the first electrode and the second electrode can provide excitation signals to all of the first piezoelectric elements 103 and the second piezoelectric elements 104, which is simple in structure. However, the embodiments are not limited thereto, and the first electrode 106 and the second electrode 107 may include a plurality of independent electrodes arranged in the axial direction.

For another example, in another feasible technical solution, as illustrated in FIGS. 4 and 5, the first piezoelectric element 103 and the second piezoelectric element 104 may be rectangular sheet structures or other sheet structures, and are alternately arranged in the axial direction to form a layered piezoelectric component. For example, two layers of piezoelectric components may be arranged, between which the first electrode 106 extending continuously in the axial direction may be disposed, i.e., the first electrode 106 is shared by the two layers of piezoelectric components. Two second electrodes 107 extending continuously in the axial direction may be respectively arranged on the outer sides of the two layers of piezoelectric components, and the polarity of the second electrode 107 is opposite to that of the first electrode 106. One layer of piezoelectric component is disposed between one of the two second electrodes 107 and the first electrode 106, and the other layer of piezoelectric component is disposed between the other of the two second electrodes 107 and the first electrode 106. The embodiments of the present disclosure, however, is not limited thereto, and the first electrode 106 and the second electrode 107 may include a plurality of independent electrodes arranged in the axial direction.

In the example illustrated in FIG. 6, the plurality of first piezoelectric elements 103 are parallel to the plurality of second piezoelectric elements 104 in the radial direction. The adjacent first piezoelectric elements 103 may be separated by an insulating element, and the adjacent second piezoelectric elements 104 may also be separated by an insulating element, so as to achieve insulation.

The first piezoelectric element 103 and the second piezoelectric element 104 may be rectangular sheet structures or other sheet structures. The plurality of first piezoelectric elements 103 are sequentially arranged at intervals in the axial direction to form one layer of first piezoelectric component (as illustrated in FIGS. 6 and 7), and the adjacent first piezoelectric elements 103 are separated by an insulating element 108 (as illustrated in FIG. 6). The plurality of second piezoelectric elements 104 are sequentially arranged at intervals in the axial direction to form one layer of second piezoelectric component (as illustrated in FIGS. 6 and 8), and the adjacent second piezoelectric elements 104 are separated by an insulating element 109 (as illustrated in FIG. 6). The first piezoelectric element 103 and the second piezoelectric element 104 are spaced apart in the radial direction. Exemplarily, the first piezoelectric element 103 and the second piezoelectric element 104 are arranged in a staggered manner in the axial direction (as illustrated in FIG. 6), so as to reduce the mutual influence therebetween. In this case, a first electrode 110 extending continuously in the axial direction may be arranged between the first piezoelectric component and the second piezoelectric component, i.e., the first electrode 110 is shared by the first piezoelectric component and the second piezoelectric component; two second electrodes 111 extending continuously in the axial direction may be respectively arranged on the outer sides of the first piezoelectric component and the second piezoelectric component, and the polarity of the second electrode 111 is opposite to that of the first electrode 110. The first piezoelectric component is disposed between one of the two second electrodes 111 and the first electrode 110, and the second piezoelectric component is disposed between the other of the two second electrodes 111 and the first electrode 110. The embodiments of the present disclosure, however, is not limited thereto, and the first electrode 110 and the second electrode 111 may include a plurality of independent electrodes arranged in the axial direction.

In some embodiments, as illustrated in FIGS. 1 and 6, the thrombolysis promoting module 10 further includes an insulating sleeve 112, and the driving module 101 and the cavitation module 102 are disposed inside the insulating sleeve 112, so that the insulating sleeve 112 separates the driving module 101 and the cavitation module 102 from the outside and achieves insulation.

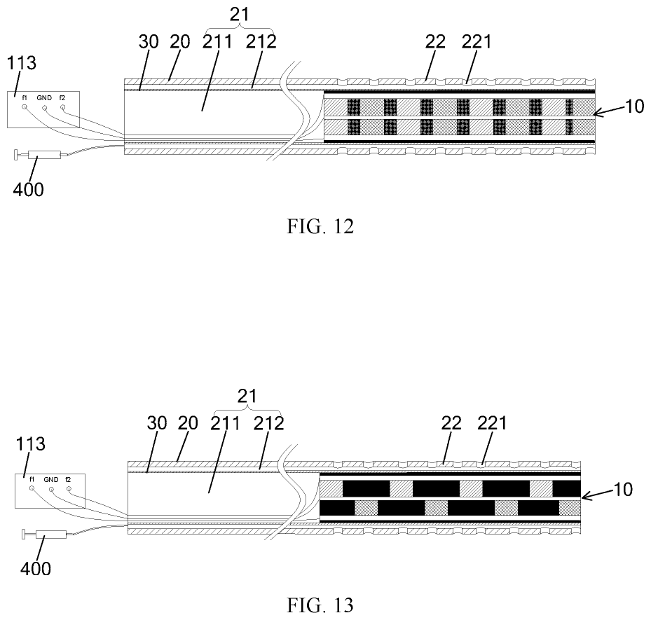

In some embodiments, as illustrated in FIGS. 12 and 13, the thrombolysis promoting module 10 further includes a control module 113, which is electrically connected to the driving module 101 and the cavitation module 102 to provide excitation signals and energy inputs to the driving module 101 and the cavitation module 102.

Specifically, the control module 113 is electrically connected to the electrodes of the driving module 101 and the electrodes of the cavitation module 102.

Embodiments of another aspect of the present disclosure provide an interventional thrombus removal device, as illustrated in FIGS. 12 and 13, which includes at least one thrombolysis promoting module 10 according to the embodiments of the first aspect. Since the structure of the thrombolysis promoting module 10 has been described in detail in the embodiments of the first aspect, the relevant content thereof is incorporated here and will not be repeated.

As illustrated in FIGS. 12 and 13, the interventional thrombus removal device further includes a main catheter 20, which defines a lumen 21 and includes a distal part 22 for accommodating the thrombolysis promoting module 10. When a patient is to be treated with interventional thrombus removal, the distal part 22 is delivered to a thrombus site in a blood vessel, and the distal part 22 is used to release microbubble precursors 100 in the lumen 21 to the outside of the main catheter 20, so that the microbubble precursors 100 and the thrombolytic drug can act on the thrombus 200 (as illustrated in FIGS. 9 and 10).

In some embodiments, the interventional thrombus removal device may include a plurality of thrombolysis promoting modules 10, which may be arranged at intervals in an axial direction of the main conduit 20.

In some embodiments, as illustrated in FIGS. 12 and 13, a side wall of the distal part 22 is provided with a through hole 221 for releasing the microbubble precursors 100. In the examples illustrated in FIGS. 12 and 13, the side wall of the distal part 22 is provided with a plurality of rows of through holes arranged at intervals in a circumferential direction, and a plurality of through holes 221 in each row are arranged at intervals in the axial direction, so that the microbubble precursors 100 and the thrombolytic drug can be released from a plurality of different positions in the circumferential direction and the axial direction.

For example, a distance between the adjacent through holes 221 in the axial direction may be 0.5 mm to 5 mm, and a diameter of each through hole may be 1 mm to 3 mm.

In some embodiments, as illustrated in FIGS. 12 and 13, the interventional thrombus removal device further includes a protective catheter 30 disposed to pass through the lumen 21, and the protective catheter 30 divides the lumen 21 into an inner central lumen 211 and an outer surrounding lumen 212, i.e., the surrounding lumen 212 surrounds the central lumen 211. The thrombolysis promoting module 10 is disposed in the central lumen 211, and the surrounding lumen 212 is in communication with the through holes 221. The surrounding lumen 212 is configured to infuse the microbubble precursors 100 and the thrombolytic drug, e.g., the microbubble precursors 100 and the thrombolytic drug are injected into the surrounding lumen 212 by a syringe 400. The protective catheter 30 is an insulating tube.

The present disclosure is described above in conjunction with specific embodiments, but it should be clear to persons skilled in the art that these descriptions are exemplary and not limiting the protection scope of the present disclosure. Persons skilled in the art can make various variations and modifications to the present disclosure according to the spirit and principle of the present disclosure, and those variations and modifications also fall within the scope of the present disclosure.

1-30. (canceled)

31. A thrombolysis promoting module, comprising:
a driving module, which is an ultrasonic module for generating acoustic energy, the driving module being configured to generate ultrasonic waves with a first frequency in a circumferential direction, and the ultrasonic waves with the first frequency being capable of driving microbubble precursors to penetrate into a thrombus;
a cavitation module, which is an ultrasonic module for generating acoustic energy, the cavitation module being configured to generate ultrasonic waves with a second frequency in the circumferential direction, the second frequency being greater than the first frequency, and the ultrasonic waves with the second frequency being capable of inducing cavitation in the microbubble precursors that penetrate into the thrombus or inducing cavitation on surfaces of the microbubble precursors to form microbubbles.

32. The thrombolysis promoting module according to claim 31, wherein the microbubble precursors are micro/nano-droplets, the first frequency is 20 kHz to 1 MHz, and the second frequency is 1 MHz to 20 MHz; or
the microbubble precursors are micro/nano-particles, the first frequency is 20 kHz to 1 MHz, and the second frequency is 1 MHz to 20 MHz.

33. The thrombolysis promoting module according to claim 31, wherein the driving module comprises one or a plurality of first piezoelectric elements, the cavitation module comprises one or a plurality of second piezoelectric elements, and the first piezoelectric element and the second piezoelectric element are insulated from each other.

34. The thrombolysis promoting module according to claim 33, wherein the plurality of first piezoelectric elements are arranged at intervals in an axial direction and insulated from each other, and the plurality of second piezoelectric elements are arranged at intervals in the axial direction and insulated from each other.

35. The thrombolysis promoting module according to claim 33, wherein the plurality of first piezoelectric elements and the plurality of second piezoelectric elements are alternately arranged in an axial direction; or
the plurality of first piezoelectric elements and the plurality of second piezoelectric elements are arranged in parallel in a radial direction.

36. The thrombolysis promoting module according to claim 33, wherein the first piezoelectric element and the second piezoelectric element are coaxially arranged in an axial direction; or
the first piezoelectric element and the second piezoelectric element are arranged in a staggered manner in the axial direction.

37. The thrombolysis promoting module according to claim 31, further comprising one of:
an insulating sleeve, inside which the driving module and the cavitation module are disposed; and
a control module electrically, which is connected to the driving module and the cavitation module to provide excitation signals and energy inputs to the driving module and the cavitation module.

38. The thrombolysis promoting module according to claim 31, wherein the thrombolysis promoting module is an interventional thrombolysis promoting module; and
the microbubble precursors penetrate into the thrombus from blood.

39. The thrombolysis promoting module according to claim 31, wherein a stage in which the driving module operates is a driving stage, a stage in which the cavitation module operates is a cavitation stage, and the driving stage and the cavitation stage are alternately carried out.

40. A thrombolysis promoting module, comprising:
a driving module, which is an ultrasonic module for generating acoustic energy, the driving module comprising one or a plurality of first piezoelectric elements;
a cavitation module, which is an ultrasonic module for generating acoustic energy, the cavitation module comprising one or a plurality of second piezoelectric elements insulated from the first piezoelectric element;
wherein the driving module is configured to generate ultrasonic waves with a first frequency in a circumferential direction, and the cavitation module is configured to generate ultrasonic waves with a second frequency in the circumferential direction, and the second frequency is greater than the first frequency.

41. The thrombolysis promoting module according to claim 40, wherein the first frequency is 20 kHz to 1 MHz, and the second frequency is 1 MHz to 20 MHz.

42. The thrombolysis promoting module according to claim 40, wherein the plurality of first piezoelectric elements are arranged at intervals in an axial direction and insulated from each other, and the plurality of second piezoelectric elements are arranged at intervals in the axial direction and insulated from each other.

43. The thrombolysis promoting module according to claim 40, wherein the plurality of first piezoelectric elements and the plurality of second piezoelectric elements are alternately arranged in an axial direction; or
the plurality of first piezoelectric elements and the plurality of second piezoelectric elements are arranged in parallel in a radial direction.

44. The thrombolysis promoting module according to claim 40, wherein the first piezoelectric element and the second piezoelectric element are coaxially arranged in an axial direction; or
the first piezoelectric element and the second piezoelectric element are arranged in a staggered manner in an axial direction.

45. The thrombolysis promoting module according to claim 40, further comprising one of:
an insulating sleeve, inside which the driving module and the cavitation module are disposed; and
a control module electrically, which is connected to the driving module and the cavitation module to provide excitation signals and energy inputs to the driving module and the cavitation module.

46. The thrombolysis promoting module according to claim 40, wherein the thrombolysis promoting module is an interventional thrombolysis promoting module.

47. The thrombolysis promoting module according to claim 40, wherein a stage in which the driving module operates is a driving stage, a stage in which the cavitation module operates is a cavitation stage, and the driving stage and the cavitation stage are alternately carried out.

48. An interventional thrombus removal device, comprising:
at least one thrombolysis promoting module; and
a main catheter defining a lumen and comprising a distal part for accommodating the thrombolysis promoting module, the distal part being configured to release microbubble precursors in the lumen to the outside of the main catheter;
wherein the thrombolysis promoting module comprises:
a driving module, which is an ultrasonic module for generating acoustic energy, the driving module being configured to generate ultrasonic waves with a first frequency in a circumferential direction, and the ultrasonic waves with the first frequency being capable of driving the microbubble precursors to penetrate into a thrombus;
a cavitation module, which is an ultrasonic module for generating acoustic energy, the cavitation module being configured to generate ultrasonic waves with a second frequency in the circumferential direction, the second frequency being greater than the first frequency, and the ultrasonic waves with the second frequency being capable of inducing cavitation in the microbubble precursors that penetrate into the thrombus or inducing cavitation on surfaces of the microbubble precursors to form microbubbles.

49. The interventional thrombus removal device according to claim 48, wherein a side wall of the distal part is provided with a through hole for releasing the microbubble precursors; and
the interventional thrombus removal device further comprises a protective catheter passing through the lumen, the protective catheter divides the lumen into a central lumen and a surrounding lumen surrounding the central lumen, the thrombolysis promoting module is arranged in the central lumen, and the surrounding lumen is in communication with the through hole.

50. The interventional thrombus removal device according to claim 48, wherein the driving module comprises one or a plurality of first piezoelectric elements; and
the cavitation module comprises one or a plurality of second piezoelectric elements insulated from the first piezoelectric element.
